(19) United States
(12) Patent Application Publication
(10) Pub. No.: US 2013/0284596 A1
(43) Pub. Date: Oct. 31, 2013

(54) Direct-transfer biopile

(71) Applicants: Abdelkader Zebda, Grenoble (FR); Alan Le Goff, Theys (FR); Michael Holzinger, Grenoble (FR); Serge Cosnier, Crolles (FR)

(72) Inventors: Abdelkader Zebda, Grenoble (FR); Alan Le Goff, Theys (FR); Michael Holzinger, Grenoble (FR); Serge Cosnier, Crolles (FR)

(73) Assignees: CENTRE NATIONAL DE LA RECHERCHE SCIENTIFIQUE, PARIS, 75 (FR); UNIVERSITE JOSEPH FOURIER, GRENOBLE, 38 (FR)

(21) Appl. No.: 13/817,536

(22) PCT Filed: Aug. 18, 2011
(86) PCT No.: PCT/FR2011/051931

§ 371 Date: Jun. 15, 2013

(30) Foreign Application Priority Data
Aug. 19, 2010 (FR) 10/56672

(51) Int. Cl. G01N 27/327

(52) U.S. Cl. CPC G01N 27/3278; G01N 27/3277; B82Y 5/00
USPC 204/403.12; 204/403.14; 29/592.1; 977/712; 977/882; 977/920

(57) ABSTRACT

The invention relates to a biopile electrode or biosensor electrode intended to be immersed in a liquid medium containing a target and an oxidizer, respectively a reducer, in which the anode comprises an enzyme able to catalyse the oxidation of a target, and the cathode comprises an enzyme able to catalyse the reduction of the oxidizer, and in which each of the anode electrode and cathode electrode consists of a solid agglomeration of carbon nanotubes mixed with the enzyme, and is secured to an electrode wire

### CROSS-REFERENCE TO RELATED APPLICATIONS

The present application is a National Stage of PCT International Application Serial Number PCT/FR2011/051931, filed Aug. 18, 2011, which claims priority under 35 U.S.C. §119 of French Patent Application Serial Number 10/56672, filed Aug. 19, 2010, the disclosures of which are incorporated by reference herein.

### FIELD OF THE INVENTION

The present invention relates to bioelectrodes adapted to biocells or to biosensors, for example, of sugar-oxygen type, for example, glucose-oxygen. The following description essentially concerns biocells. It should be understood that a biosensor has the same structure as a biocell but is used to detect the content of one of the components of the enzymatic reaction, for example, glucose.

### DISCUSSION OF PRIOR ART

Various types of glucose-oxygen biocells are described in prior art. For example, in patent application PCT/FR2009/050639 (B8606), each electrode, anode and cathode, of the biocell corresponds to a chamber containing a liquid medium having an electrode wire plunged therein. The anode and cathode chambers are delimited by membranes capable of being crossed by hydrogen and oxygen but avoiding the circulation of other heavier elements.

The anode comprises, in a solution, an enzyme and a redox mediator. The enzyme is capable of catalyzing the oxidation of sugar and for example is glucose-oxidase if the sugar is glucose. The redox mediator has a low redox potential capable of exchanging electrons with the anode enzyme and for example is ubiquinone (UQ).

The cathode also comprises, in a solution, an enzyme and a redox mediator. The enzyme is capable of catalyzing the reduction of oxygen and for example is polyphenol oxidase (PPO). The redox mediator has a high redox potential capable of exchanging electrons with the cathode enzyme and for example is hydroquinone (QHD).

Reactions of the following type then occur at the anode and at the cathode:

Cathode: QH2+½O2Q+H2O

Anode: glucose+UQ gluconolactone+UQH2

Cathode: Q+2H++2e−QH2

Anode: UQH2UQ+2H++2e−

A 20-mV anode potential and a 250-mV cathode potential are then obtained, which results in a 230-mV zero-current potential difference of the biocell.

Such biocells operate properly but require for anode and cathode conductors to be plunged into chambers containing appropriate liquids, which is a practical disadvantage in many cases and especially makes it very difficult, or even impossible, to implant such biocells in living beings, especially to power various actuators, such as heart stimulators, artificial sphincters, or even artificial hearts.

Unpublished French patent application 10/52657 of Apr. 8, 2010 describes such a biocell. As illustrated in FIG. 1, this biocell comprises an anode body A and a cathode body K. The anode body is formed of a solid body comprising a conductive material associated with an appropriate anode enzyme and redox mediator. The anode body has a fixedly attached anode wire 1. Similarly, the cathode is formed of a solid body formed of a conductor associated with an appropriate enzyme and cathode mediator. The cathode body has a fixedly attached cathode wire 3. The anode and cathode wires, for example, made of platinum, are shown as penetrating into the anode and cathode bodies; they may be simply bonded to these bodies. The anode body and the cathode body are for example formed by compression of powder graphite mixed with the appropriate enzyme and redox mediator.

Chemical redox mediators enable to provide an electric connection between the enzyme and the electrode by electron jumping between the redox mediators positioned between the electrode surface and the prosthetic center or active center of the enzyme.

Besides the complexification of the construction of bioelectrodes (redox mediators being generally soluble in an aqueous medium, it is necessary to fix them on the electrode surface), a main disadvantage of the use of such mediators is the fact that they strongly decrease the potential provided by the biocell. By definition, such mediators must have a voltage greater than that of the redox center of the enzyme catalyzing the glucose oxidation to be able to react therewith, in particular with its reduced form, in order to oxidize it. Similarly, redox mediators dedicated to the connection of the oxygen-reducing enzyme must have a lower potential than the active center of this enzyme to be able to react with its oxidized form. As a result, the potential difference between the active sites of the enzyme catalyzing the oxidation of glucose and that catalyzing the reduction of oxygen is necessarily greater than the potential difference between the redox mediators implied in these two reactions. For example, theoretically, a glucose/oxygen biocell should provide a 1-V potential, or the use of redox mediators results in biocells having much lower potentials. It should be noted that the cell potential is also limited by problems of kinetic limitation and ohmic drop.

It has thus been attempted to perform a direct electric connection (without using mediators) of the enzymes to the electrodes. However, the electron transfer remains very low and sometimes requires modifying the enzyme. Further, the efficiency of such cells remains low.

High-efficiency implantable biocells are thus needed.

### SUMMARY

Thus, according to an embodiment of the present invention, bioelectrodes which are simple to handle for applications in the field of biocells and of biosensors, and that can in particular be implanted in an animal or human living being, are desired to be formed.

More specifically, an embodiment of the present invention provides a biocell or biosensor electrode intended to be immersed in a liquid medium containing a target and an oxidizer, respectively a reducer, wherein the anode comprises an enzyme capable of catalyzing the oxidation of a target, and the cathode comprises an enzyme capable of catalyzing the reduction of the oxidizer, and wherein each of the anode and cathode electrodes is formed of a solid concretion of carbon nanotubes mixed with the enzyme, and has an electrode wire fixedly attached thereto.

According to an embodiment of the present invention, the electrode is surrounded with a semipermeable membrane letting through the oxidizer and the target and blocking the enzyme.

According to an embodiment of the present invention, the membrane is of dialysis membrane type.

According to an embodiment of the present invention, this target is glucose.

An embodiment of the present invention provides a method for manufacturing a biocell or a biosensor, wherein the anode and the cathode are formed by compression of a mixture in solution comprising carbon nanotubes and an enzyme.

According to an embodiment of the present invention, the carbon nanotubes are of multiple-walled type.

### BRIEF DESCRIPTION OF THE DRAWINGS

The foregoing and other features and advantages will be discussed in detail in the following non-limiting description of specific embodiments in connection with the accompanying drawings, among which:

FIG. 1 very schematically shows a biocell with solid electrodes;

FIG. 2 illustrates the current and power performance according to the voltage of a glucose/oxygen biocell formed of bioelectrodes manufactured according to the present invention; and

FIGS. 3A and 3B respectively illustrate the electrochemical response of a biosensor to glucose injections and the current response of this biosensor according to the glucose concentration.

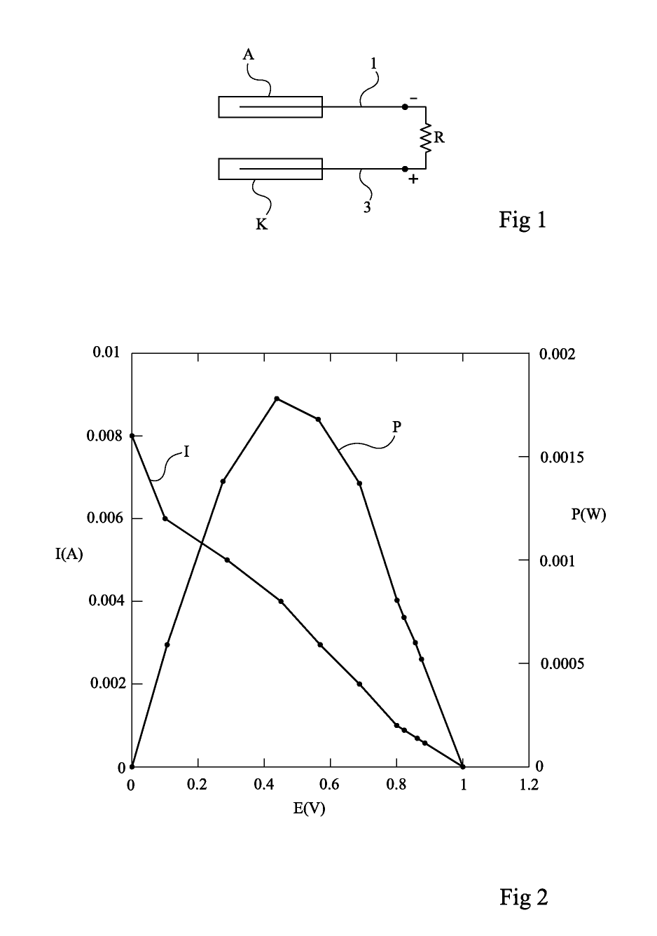

### DETAILED DESCRIPTION

Generally, the present invention relates to a new type of solid electrode containing electrically-connected enzymes. The invention provides the electric connection of a large density of enzymes by compression in the form of a compact block, for example, a disk, of a mixture of carbon nanotubes, of enzymes, of water necessary to solubilize the enzymes, and of glycerol as a binder between the different components. Carbon nanotubes are of single- or multiple-walled type.

The use of very thin and very conductive carbon nanotubes enables to fix the enzymes and to connect them, due to the fact that carbon nanotubes penetrate into the large enzyme molecules which are formed of a protein envelope protecting their active redox center.

The conductivity of the nanotubes and their very small diameter (on the order of one nanometer) allows an electric communication with the enzyme which keeps its catalytic activity. The catalytic properties of the enzyme may be exploited either for bioelectrochemical detection, or for energy conversion, and more specifically the production of electric energy. Such bioelectrodes may be used in the fields of biocells and of biosensors.

The anode is for example formed of a compression of carbon nanotubes containing an oxidase such as glucose oxidase (GOX) capable of catalyzing the oxidation of a fuel, for example, glucose.

The cathode is for example formed of a compression of carbon nanotubes containing an enzyme such as laccase or bilirubin oxidase capable of catalyzing the reduction of an oxidizer such as oxygen.

Reactions of the following type then occur at the anode and at the cathode:

Cathode: ½O2 2e+H2O

Anode: −2e−2H+gluconolactone

Such reactions are given in the specific case where the fuel is glucose, the anode enzyme is glucose-oxidase (GOX), and the oxidizer is oxygen. The cathode enzyme is laccase.

As an example, an anode has been prepared by mixing 150 mg of carbon nanotubes, 30 mg of glucose oxidase, and 30 mg of catalase (the function of catalase is to eliminate H2O2 (a noxious product) formed by glucose oxidase in the presence of O2: H2O2®½O2+H2O), 0.6 ml of water and glycerol (50 μl) in a ceramic mortar. A cathode has been prepared in similar fashion: 150 mg of carbon nanotubes, 30 mg of laccase, 0.6 ml of water, and 25 μl of glycerol have been mixed in a ceramic mortar. The resulting pastes formed of carbon nanotubes and enzymes have been compressed at a 1,500-kg/cm2 pressure to form disks. The surface and the thickness of the disks were respectively 1.33 cm2 and 0.1 cm. A platinum wire has been attached with a conductive glue to the compacted carbon nanotubes on one side of each disk and covered with a silicon film to reinforce the mechanical strength of the biomaterial and the electric contact.

To operate as a cell, these anode and cathode bodies are placed in a fluid containing oxygen and a sugar, for example, glucose.

This biocell has a 1-V zero-current potential, a 1,800-μW/cm2 maximum power, and a 8-mA maximum current. Such a performance is much higher than those obtained for known biocells with a direct enzyme connection (5-μW/cm2 maximum power and 0.73-V maximum zero-current potential). Further, this biocell gives the possibility of having a significant power at a sufficiently high potential to actuate devices: 800 μW at 0.8 V.

FIG. 2 shows curves of power and electric current according to the potential of a biocell such as described hereabove as an example.

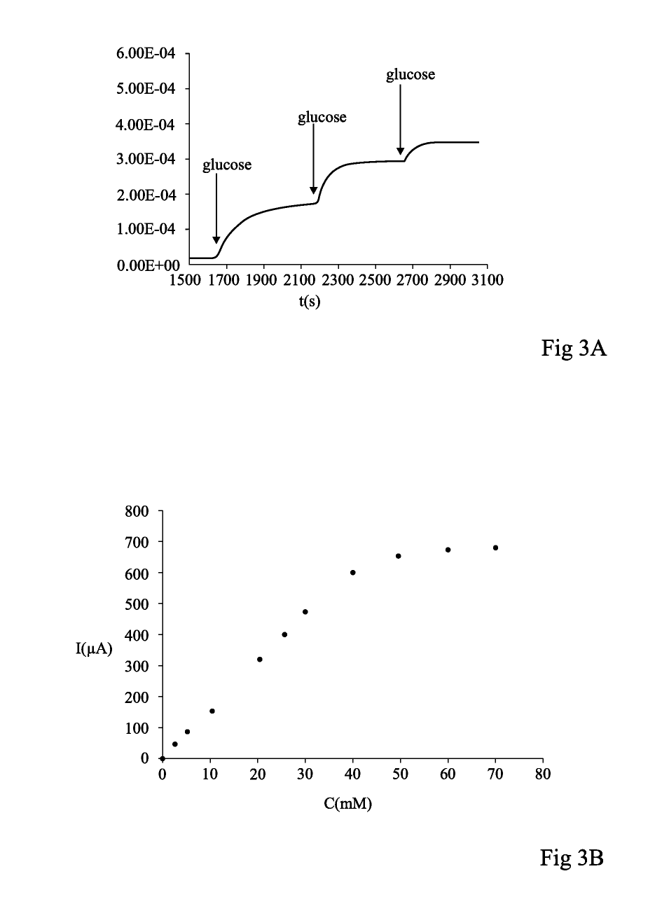

FIG. 3A illustrates the electrochemical response of a biosensor constructed like the above-described cell to the presence of glucose and FIG. 3B shows the measured current according to the glucose concentration.

To use this biosensor, the electrode is plunged into an aqueous liquid and glucose is added. An electric potential, for example, 0.1 V, is applied between the bioelectrode and a reference electrode, both plunged into the liquid analysis medium and the electric current is measured between the bioelectrode and an auxiliary electrode immersed in this medium. The detection and the quantification of the glucose present in the liquid are performed by measurement of the current of the glucose oxidation catalyzed by the enzyme.

The performance of the biosensor maintained at the 0.1-V potential are 17 mA/M/cm2 and 685 μA/cm2 respectively for the sensitivity and the maximum current density. In addition to a work potential enabling to do away with anode interferences, this system has the strongest maximum current density described up to date, even for conventional glucose biosensors.

However, it has been observed that biocells using such anode and cathode bodies have a short lifetime. The present inventors have imputed this problem to the fact that enzyme leaks along time out of the anode body and of the cathode body. To solve this problem, each of the anode and cathode bodies may be surrounded with a microperforated membrane such as membranes currently used in dialysis, which let through glucose and oxygen and prevent the passing of the enzyme and of carbon nanotubes of greater molecular weight. The anode and cathode electrodes may altogether be surrounded with a semipermeable membrane letting through glucose and oxygen and blocking enzymes and carbon nanotubes, especially to enable their implantation in an animal or human body.

The example of a glucose-oxygen biocell has been given hereabove. Any sugar-oxygen biocell may be modified according to the present invention, and more generally any biocell having its anode comprising an enzyme capable of catalyzing the oxidation of a target, and having its cathode comprising an enzyme capable of catalyzing the oxidizer reduction.

Various embodiments with different variations have been described hereabove. It should be noted that those skilled in the art may combine various elements of these various embodiments and variations without showing any inventive step.

1. A biocell or biosensor electrode intended to be immersed in a liquid medium containing a target and an oxidizer, respectively a reducer, wherein the anode comprises an enzyme capable of catalyzing the oxidation of a target, and the cathode comprises an enzyme capable of catalyzing the reduction of the oxidizer, and wherein each of the anode and cathode electrodes is formed of a solid concretion of carbon nanotubes mixed with the enzyme, and has an electrode wire fixedly attached thereto.

2. The biocell or biosensor of claim 1, wherein the anode and cathode electrodes are surrounded with a semipermeable membrane letting through the oxidizer and the target and blocking the enzyme.

3. The biocell or biosensor of claim 2, wherein said membrane is of dialysis membrane type.

4. The biocell or biosensor of claim 1, wherein the target is glucose.

5. A method for manufacturing a biocell or a biosensor, wherein the anode and the cathode are formed by compression of a mixture in solution comprising carbon nanotubes and an enzyme, excluding any redox mediator.

6. The method of claim 5, wherein the carbon nanotubes are of multiple-walled type.

7. The biocell or biosensor of claim 2, wherein the target is glucose.
